(19) United States
(12) Patent Application Publication
(10) Pub. No.: US 2021/0331139 A1
(43) Pub. Date: Oct. 28, 2021

(54) POROUS CERAMIC BODIES INCLUDING ALUMINA MESOCRYSTALS

(71) Applicant: Scientific Design Company, Inc., Little Ferry, NJ (US)

(72) Inventors: Wojciech L. Suchanek, Wyckoff, NJ (US); Michael Di Mare, Morristown, NJ (US); Jean R. Adam, Roselle, NJ (US); Nicholas D'Angelo, Glen Rock, NJ (US)

(73) Assignee: Scientific Design Company, Inc., Little Ferry, NJ (US)

(21) Appl. No.: 17/240,372

(22) Filed: Apr. 26, 2021

Related U.S. Application Data
(60) Provisional application No. 63/016,013, filed on Apr. 27, 2020.

(51) Int. Cl. B01J 21/04; B01J 23/68; B01J 37/00; B01J 37/08; B01J 37/02; B01J 35/10; C07D 301/00

(52) U.S. Cl. CPC B01J 21/04; B01J 23/688; B01J 37/0009; B01J 37/088; B01J 37/0215; C07D 301/00; B01J 35/1014; B01J 35/1076; B01J 35/1071; B01J 35/1066; B01J 35/1042

(57) ABSTRACT

A porous ceramic body is provided for a variety of applications. The porous ceramic body includes mesocrystals of alumina such as, for example, alpha alumina. Porous alpha alumina bodies containing the mesocrystal microstructure can provide enhanced activity and catalyst lifetime when the same is used as a carrier for a silver-based ethylene oxide catalyst.

### CROSS REFERENCE TO RELATED APPLICATIONS

The present invention claims priority to U.S. Provisional Patent Application No. 63/016,013, filed Apr. 27, 2020, the entire content and disclosure of which is incorporated herein by reference.

### FIELD OF THE INVENTION

The present invention relates to porous ceramic bodies, and more particularly to porous alumina bodies that can be used in a wide variety of applications including, for example, as an insulator, a refractory, a filler, an abrasive, a substrate, a filter, a membrane, or a catalyst carrier. In some embodiments, the present invention provides a porous alpha alumina body that can provide extended lifetime to a silver-based ethylene oxide (EO) catalyst supported on the same.

### BACKGROUND

In the chemical industry and the chemical engineering industry, reliance is oftentimes made on using porous ceramic bodies which are capable of performing or facilitating separations or reactions and/or providing areas for such separations and reactions to take place. Examples of separations or reactions include: filtration of gases and liquids, adsorption, reverse osmosis, dialysis, ultrafiltration, or heterogeneous catalysis. Although the desired physical and chemical properties of such porous ceramic bodies vary depending on the particular application, there are certain properties that are generally desirable in such porous ceramic bodies regardless of the final application in which they will be utilized.

For example, porous ceramic bodies may be substantially inert so that the porous ceramic bodies themselves do not participate in the separations or reactions taking place around, on or through them in a way that is undesired, unintended, or detrimental. In applications where it is desired to have the components that are being reacted or separated pass through, or diffuse into, the porous ceramic body, a low diffusion resistance (e.g., high effective diffusivity) would be advantageous.

In some applications, the porous ceramic bodies are provided within a reaction or separation space, and so they are desirably of high pore volume and/or high surface area, in order to improve the loading and dispersion of the desired reactants, and also to provide enhanced surface area on which the reactions or separations can take place. These applications also require sufficient mechanical integrity to avoid being damaged, i.e., crushed, chipped or cracked, during transport or placement.

In some applications, the porous ceramic body can be used as a carrier (or support) for a silver-based EO catalyst that is used in EO production. In EO applications, there is a constant need for longer life EO catalysts. One of the methods to increase catalyst lifetime is to reduce catalyst aging, which can be achieved by reducing operating temperature of the EO catalyst. It has been reported that EO catalysts exhibiting higher activity, i.e., operate at lower temperatures, can have extended lifetime. Despite this method of increasing catalyst lifetime, there is a need for providing other means that can provide EO catalysts with extended catalyst lifetime.

### SUMMARY

A porous ceramic body is provided that is capable of performing or facilitating separations, or performing reactions and/or providing areas for such separations or reactions to take place. In one embodiment of the present invention, the porous ceramic body includes mesocrystals of alumina. The term “porous ceramic body” denotes a structure that is larger than an agglomerate of particles of a powder. The term “mesocrystals” denotes a “nanostructured material showing clear evidence that it consists of individual nanoparticle building units with a defined order on the atomic scale in at least one direction, which can be inferred from the existence of an essentially sharp wide angle diffraction pattern”, as defined by E. V. Sturm and H. Colfen [“Review: Mesocrystals: Past, Presence, Future”, Crystals 2017, vol. 7, p. 207; doi:10.3390/cryst7070207]. The term mesocrystals is an abbreviation for ‘mesoscopically structure crystal’, where individual subunits often form a perfect 3D order or a mosaic structure. With a variety of mesocrystal forms, level of faceting of individual subunits may vary [“Review: Mesocrystals: Past, Presence, Future”, Crystals 2017, vol. 7, p. 207; doi:10.3390/cryst7070207]. The only alumina mesocrystals reported so far exhibited round surfaces and formed worm-like well-branched structures without characteristic crystallographic facets of individual subunits [J. Am. Ceram. Soc., Vol. 93, No. 2, pp. 399-412 (2010)]. Stated in other terms, the present invention provides a porous ceramic body including alumina crystallites that do not contain well-defined crystallographic faceting. The term “alumina” is defined herein to include all aluminum oxides, hydrated oxides of aluminum, aluminum hydroxides, and aluminum oxide-hydroxides.

In some embodiments, a porous ceramic body is provided that includes alumina crystallites without well-defined crystallographic facets.

The porous ceramic body of the present invention can be used in a wide variety of applications such as, for example, as an insulator, a refractory, a filler, an abrasive, a substrate, a filter, as a membrane or as a catalyst carrier. In one example, the porous ceramic body of the present invention can be used as a carrier for a catalytically active material. In one specific embodiment of the present invention, a porous alpha alumina carrier is provided that can be used as a carrier for a silver-based EO catalyst. In such an embodiment, the silver-based EO catalyst includes a carrier including at least 80% alpha alumina, wherein the alpha alumina contains alpha alumina mesocrystals. The EO catalyst further includes a catalytic amount of silver disposed on and/or in the carrier, and a promoting amount of one or more promoters disposed on the carrier. Such a silver-based EO catalyst that includes a carrier that contains mesocrystals of alpha alumina unexpectedly has higher catalytic activity and improved catalyst lifetime as compared to an equivalent EO catalyst in which the carrier does not contain mesocrystals of alpha alumina.

### BRIEF DESCRIPTION OF THE DRAWINGS

FIGS. 1A-1F show different crystal faceting of alpha alumina.

FIGS. 2A-2B are scanning electron microscopy (SEM) pictures of a comparative porous alpha alumina body, CPB1, that contains well-defined crystallographic faceting.

FIGS. 3A-3B are scanning electron microscopy (SEM) pictures of a porous alpha alumina body, PB1, in accordance with the present invention.

FIGS. 4A-4B are scanning electron microscopy (SEM) pictures of a porous alpha alumina body, PB2, in accordance with the present invention.

FIGS. 5A-5B are scanning electron microscopy (SEM) pictures of a porous alpha alumina body, PB3, in accordance with the present invention.

FIGS. 6A-6B are scanning electron microscopy (SEM) pictures of a porous alpha alumina body, PB4, in accordance with the present invention.

FIG. 7 is a transmission electron microscopy (TEM) image of comparative porous alpha alumina body, CPB1, with an insert including the electron diffraction pattern of CPB1.

FIG. 8 is a transmission electron microscopy (TEM) image of porous alpha alumina body, PB1, in accordance with the present invention, with an insert including the electron diffraction pattern of PB1.

FIG. 9 is a transmission electron microscopy (TEM) image of porous alpha alumina body, PB2, in accordance with the present invention, with an insert including the electron diffraction pattern of PB2.

FIG. 10 is a transmission electron microscopy (TEM) image of porous alpha alumina body, PB4, in accordance with the present invention, with an insert including the electron diffraction pattern of PB4.

FIG. 11 is a transmission electron microscopy (TEM) image of porous alpha alumina body, PB7, in accordance with the present invention, with an insert including the electron diffraction pattern of PB7.

### DETAILED DESCRIPTION

The present invention will now be described in greater detail by referring to the following discussion and drawings that accompany the present invention. In the following description, numerous specific details are set forth, such as particular structures, components, materials, dimensions, processing steps and techniques, in order to provide an understanding of the various embodiments of the present invention. However, it will be appreciated by one of ordinary skill in the art that the various embodiments of the present invention may be practiced without these specific details. As used throughout the present invention, the term “about” generally indicates no more than ±10%, ±5%, ±2%, ±1% or ±0.5% from a number.

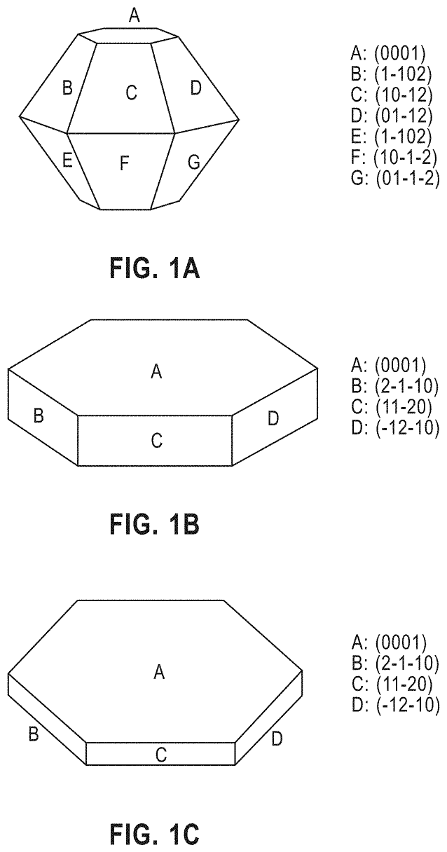

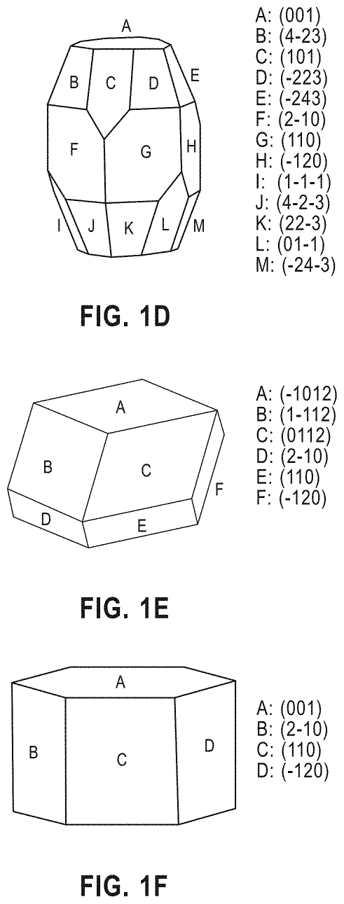

Alpha alumina (α-Al₂O₃, corundum) is a very well-known material. Crystals of alpha alumina exhibit several types of equilibrium and non-equilibrium faceting types [J. Am. Ceram. Soc., Vol 81, No. 6, pp. 1411-1420 (1998), J. Am. Ceram. Soc., Vol 79, No. 1, pp. 88-96 (1996)]. They include equiaxed, elongated or platy structures, examples of which are shown in FIGS. 1A, 1B, 1C, 1D, 1E and 1F. One characteristic feature of the faceted alpha alumina crystals is that each flat surface corresponds to a particular crystallographic orientation, which is also marked for particular facets/crystals in FIGS. 1A, 1B, 1C, 1D, 1E and 1F.

Typical EO carriers or other porous bodies are made of alpha alumina powders that have well-defined crystallographic faceting. Each grain (or crystallite) of an alpha alumina powder is a small alpha alumina crystal with very specific faceting. The crystallites are randomly oriented with respect to each other with well-defined and visible grain boundaries. Grain boundary can be defined here as a border between two alpha alumina regions with different crystallographic orientations, i.e., crystallites. EO carriers or other porous bodies including such well-faceted alpha alumina crystallites are available from commercial suppliers. EO carriers or other porous bodies made from alpha alumina powders that contain well-defined crystallographic faceting do not contain mesocrystals of alpha alumina.

However, and as will be described herein, the well-faceted morphology of alpha alumina crystallites is not necessary for EO carriers or other porous bodies. The present invention discloses the synthesis and properties of porous ceramic bodies that include alumina crystallites without well-defined crystallographic faceting; the term “alumina”, as defined previously, includes all aluminum oxides, hydrated oxides of aluminum, aluminum hydroxides, and aluminum oxide-hydroxides. Such porous ceramic bodies include mesocrystals of alumina. When a porous ceramic body including mesocrystals of alpha alumina is used as an EO carrier, the silver-based catalyst that is supported on such a carrier was surprisingly found to increase activity of silver-based catalyst during EO production.

Mesocrystals of various metal oxides have been reported [see, for example, U.S. Pat. No. 8,865,116 B2; U.S. Patent Application Publication No. 2015/0034150 A1; WO 2017/168126 A1; and U.S. Patent Application Publication No. 2018/0147566 A1] including alpha alumina [J. Am. Ceram. Soc., Vol. 93, No. 2, pp. 399-412 (2010)]. However, none of the existing publications known to the Applicant even remotely relates to porous ceramic bodies, EO supports, EO catalysts or EO catalysis.

Alpha alumina crystallites can possess round surfaces and form worm-like well-branched structures without characteristic crystallographic facets of individual crystallites [J. Am. Ceram. Soc., Vol. 93, No. 2, pp. 399-412 (2010)]. They substantially differ from the well-faceted alpha alumina based on crystallite morphology. There is no well-established relationship between shape of such non-faceted alpha alumina crystallites and any particular crystallographic direction.

It should be noted that the crystallinity of such non-faceted alpha alumina crystallites is exactly the same as of the well-faceted alpha alumina crystallites. This can be seen by X-ray diffraction analysis. Thus the only difference between both types of alpha alumina crystallites is their morphology type (non-faceted vs. faceted).

In general terms, the present invention provides a porous ceramic body that includes mesocrystals or non-faceted crystallites of alumina. The alumina that is present in the porous ceramic body of the present invention can include, but is not limited to, alpha alumina (α-Al₂O₃), gamma alumina (γ-Al₂O₃), beta alumina (β-Al₂O₃), theta alumina (Θ-Al₂O₃), delta alumina (δ-Al₂O₃), chi alumina (χ-Al₂O₃), rho alumina (ρ-Al₂O₃), eta alumina (η-Al₂O₃) transition aluminas, akdalaite (5Al₂O₃.H₂O), tohdite (5Al₂O₃.H₂O), boehmite (γ-AlOOH), pseudo-boehmite (AlOOH), diaspore (α-Al(OH)₃), gibbsite (α-Al(OH)₃), hydrargillite (α-Al(OH)₃), bayerite (β-Al(OH)₃), doyleite (Al(OH)₃), nordstrandite (Al(OH)₃), amorphous aluminas, and mixtures thereof. In one preferred embodiment, the alumina that is present in the porous ceramic body is alpha alumina; in such an embodiment, a porous alpha alumina body is provided. In one embodiment of the present invention, the alpha alumina body of the present invention includes at least 80% alpha alumina crystallites in which at least 5%, preferably at least 10%, more preferably at least 50%, and even more preferably at least 75%, of the alpha alumina crystallites are in the form of mesocrystals. Further details regarding the porous alpha alumina body will be provided herein below.

The porous ceramic body of the present invention that includes mesocrystals or non-faceted crystallites of alumina can have a variety of properties including, for example, pore volume, water absorption, B.E.T. surface area, porosity, average crush strength, attrition value, and etc., that can vary depending on the materials (i.e., powders, burnout materials and binders) used in forming the porous ceramic body. The physical properties of the resultant porous ceramic bodies of the present invention can be tailored for a specific end use of the porous ceramic body.

The porous ceramic body of the present invention can have non-faceted alumina crystallites forming worm-like and well-branched structures. Grain boundaries are in some cases visible, but less frequent than a porous ceramic body having faceted alumina crystallites. The size of the crystallites, their connectivity and shapes can vary with the method used to form the same. Detailed TEM analysis can be used to confirm the lack of clear faceting and grain boundaries within small regions of the crystallites. Fractured surfaces can be present in the porous ceramic body of the present invention.

In general, the porous ceramic body of the present invention can be prepared by (i) providing a precursor mixture, (ii) forming the precursor mixture into a desired shape, and (iii) subjecting the shaped precursor mixture to a heat treatment process. The precursor mixture of the present invention provides mesocrystal porous ceramic bodies after the heat treatment process. The various processing steps mentioned above will now be described in greater detail.

In one embodiment the precursor mixture can be made by first adding, in any order, all the dry/powder components (i.e., alumina power(s), burnout material(s) and binder(s)) to a mixer/blender (or other apparatus in which mixing/blending can be performed). The dry/powder components are mixed to provide a homogenous dry mixture. The term “homogeneous,” as used herein, indicates that individual macroscopic regions of agglomerated particles (i.e., of at least 100 or 200 microns) of each substance in the dry mixture are typically not detectable or present in the homogeneous dry mixture, although individual microscopic regions of agglomerated particles (e.g., less than 100 or 200 microns), may or may not be present. Liquid components including, for example, solvents and lubricants are added to the homogeneous dry mixture, and the mixture including the liquid components and homogeneous dry mixture are mixed to provide a precursor mixture. The above method of forming the precursor mixture can be referred to herein as a ‘first precursor mixture preparation process’.

In another embodiment, the precursor mixture can be made by first adding at least some of dry/powder components to a mixer/blender (or any other apparatus in which mixing/blending can be performed) that includes a liquid, e.g., water. The dry/powder components and the liquid are then mixed to provide a first mixture. If needed, other dry/power components/powders can be added to the first mixture and mixed to provide a second mixture of all dry/powder components and liquid. Next, solvents and lubricants are added to the first or second mixtures, and these components are mixed to provide the precursor mixture. The above method of forming the precursor mixture can be referred to herein as a ‘second precursor mixture preparation process’.

Although the present invention describes and illustrates two methods of forming the precursor mixture, one skilled in the art will immediately recognize that other variants of the steps and procedures described above are possible, and/or the order of adding the various components can be changed based on the equipment used in providing the precursor mixture.

The dry/powder components that can be used in providing the precursor mixture of the present invention can include one or more alumina powders, one or more burnout materials and, optionally one or more binders. The one or more alumina powders can include, but are not limited to, alpha alumina (α-Al₂O₃), gamma alumina (γ-Al₂O₃), beta alumina (β-Al₂O₃), theta alumina (Θ-Al₂O₃), delta alumina (δ-Al₂O₃), chi alumina (χ-Al₂O₃), rho alumina (ρ-Al₂O₃), eta alumina (η-Al₂O₃) transition aluminas, akdalaite (5Al₂O₃.H₂O), tohdite (5Al₂O₃.H₂O), boehmite (γ-AlOOH), pseudo-boehmite (AlOOH), diaspore (α-Al(OH)₃), gibbsite (α-Al(OH)₃), hydrargillite (α-Al(OH)₃), bayerite (β-Al(OH)₃), doyleite (Al(OH)₃), nordstrandite (Al(OH)₃), amorphous aluminas, and mixtures thereof. The one or more alumina powders employed in the present invention are composed of particles that do not have a well-defined crystallographic faceting. Such powders can be made using techniques well known to those skilled in the art or they can be commercially purchased.

In one embodiment and for forming a porous alpha alumina body, the alumina powder includes gibbsite. In another embodiment and for forming a porous alpha alumina body, the alumina powder includes gibbsite as the major powder component, and alpha alumina as the minor powder component. In a further embodiment and for forming a porous alpha alumina body, the alumina powder includes boehmite. In a yet further embodiment and for forming a porous alpha alumina body, the alumina powder includes boehmite as the major powder component, and alpha alumina as the minor powder component. In even a further embodiment, and in forming a porous alpha alumina body, the alumina power includes gibbsite and boehmite as major powder components, and alpha alumina as the minor powder component. In yet another embodiment, alpha alumina is the major powder component.

In forming a porous alpha alumina body, the alumina powder(s) that is(are) used can be characterized by an average or median particle size (e.g., D₅₀, the particle size where half of the particle population lies below the indicated value) in a range of 0.1 to 100 microns, and preferably 0.25 to 50 microns. In some embodiments, alumina powder(s) that is(are) used has a very high purity, i.e., about 95 or 98 wt % or more. The particle sizes given above can refer to a diameter for the case where the particle is spherical or approximately spherical. For cases where the particles substantially deviate from a spherical shape, the particle sizes given above are based on the equivalent diameter of the particles. As known in the art, the term “equivalent diameter” is used to express the size of an irregularly-shaped object by expressing the size of the object in terms of the diameter of a sphere having the same volume as the irregularly-shaped object.

The amount of alumina powder(s) that is(are) present in the dry precursor mixture (solids only, no solvents) is typically from about 35% to about 95% by weight, and more specifically between about 60% and about 90% by weight. In embodiments, when alpha alumina is used as a minor powder component, the amount of alpha alumina that is present in the precursor mixture is from about 0.1% to about 20% by weight of the total weight of alumina components in the precursor mixture, and more specifically between about 1% and about 10% by weight, while the amount of the majority power component is from about 80% to about 99.9% by weight, and more specifically between about 90% and about 99% by weight of the total weight of alumina components in the precursor mixture.

The one or more burnout materials, which also may be referred to a temporary binder, that can be used in the present invention includes any of the burnout materials known in the art, such as granulated polyolefins (e.g., polyethylene or polypropylene), carbons (e.g., graphite, amorphous carbon, activated carbon, carbon black, carbon coke, etc.) cellulose, substituted celluloses (e.g., methylcellulose, ethylcellulose, and carboxyethylcellulose), stearates (such as organic stearate esters, e.g., methyl or ethyl stearate), starches (such as corn starch, potato starch, etc.), waxes, walnut shell flour, and the like, which are decomposable at the temperatures employed. The burnout material is primarily responsible for imparting porosity to the porous ceramic body, and to ensure the preservation of a porous structure during the green (i.e., unfired phase) in which the mixture may be shaped into particles by molding or extrusion processes. Burnout materials are generally substantially or completely removed during firing to produce the finished porous ceramic body. The burnout material may have a D₅₀ particle size in a range of about, for example, 0.05-100 microns, preferably 0.1-60 microns, and more preferably 0.3-45 microns.

In some embodiments, the burnout material is a mixture of graphite and other non-graphite carbon powder(s) (e.g., amorphous carbon, activated carbon, carbon black, carbon coke, etc., or a mixture thereof). They can have the same or different particle sizes (i.e., D₅₀ particle size), and they can be added simultaneously or sequentially. In some embodiments, the non-graphite carbon is included in an amount by weight greater than the amount by weight of graphite. For example, the weight ratio of non-graphite powder to graphite may be 0.1:1 to about 10:1, and preferably 0.25 to about 4. In other embodiments, only graphite powder is employed; in such an embodiment the non-graphite powder(s) is(are) essentially absent. In some other embodiments, there is no graphite powder.

In some embodiments of the present invention, burnout materials that have a decomposition temperature of 500° C. or greater (i.e., a high-temperature burnout material) are excluded from being used, and only burnout materials that have a decomposition of less than 500° C. (i.e., a low-temperature burnout material) are used. The exclusion of a high-temperature burnout material from the precursor mixture “unplugs” the pores at low temperatures and thus facilitates gas transport during the oxidation stage to enhance the oxidation kinetics of the burnout material. In one embodiment, the low-temperature burnout material that can be used in the present invention has a decomposition temperature from 100° C. to 500° C. Exemplary low-temperature burnout materials that can be used in the present invention include, but are not limited to, granulated polyolefins (e.g., polyethylene and polypropylene), Vaseline®, petroleum jelly, waxes, starches, polymers, plastics, oils, and other natural or artificial organic compounds and materials. In some embodiments, a single low-temperature burnout material such as, for example, granulated polyethylene, is employed. In other embodiments, a combination of at least two low-temperature burnout materials such as, for example, granulated polyethylene and corn starch, can be employed. In embodiments in which a mixture of low-temperature burnout materials is employed, it may be preferred, in some instances, to use a greater amount of the lowest low-temperature burnout material as compared to a higher low-temperature burnout material. The amount of the low-temperature burnout material that is present in the precursor mixture is typically from about 1% to about 55% by weight, more preferentially between about 5% and about 35% by weight.

In general, the amount of the burnout material that is present in the dry precursor mixture (solids only, solvents not included) is typically from about 5% to about 65% by weight, and more preferentially between about 10% and about 40% by weight.

The one or more binders or sintering aids can include permanent binders such as, for example, inorganic clay-type materials, such as silicas, silicates, and an alkali or alkali earth metal compound. A convenient binder or sintering aid material which may be incorporated with the alumina powders comprises a stabilized silica sol, and optionally alkali or alkali earth metal salt. In some embodiments, a silicon-containing substance is substantially or completely excluded from the method for producing the porous ceramic body. In the case of a silicon-containing substance being substantially excluded from the porous ceramic body, a trace amount of silicon derived from impurities in the raw materials used to prepare the porous ceramic body may still be present in the porous ceramic body. Such trace amounts are generally no more than 1%, 0.5%, or 0.1% by weight of the porous ceramic body. The amount of the binder or sintering aid material that is present in the precursor mixture is typically from about 0.0% to about 5.0% by weight, and more specifically between about 0.0% and about 2.0% by weight.

The solvents and lubricants that can be employed in forming the precursor mixture of the present invention include conventional solvents and lubricants that are well known to those skilled in the art. For example, the solvent used in forming the precursor mixture can include water, and the lubricant can include petroleum jelly. In some embodiments, the lubricants can be omitted from being used. When employed, the amount of lubricant that is present in the precursor mixture is typically from about 1% to about 20% by weight, more specifically between about 2% and about 15% by weight. The amount of solvent that is present in the precursor mixture is typically from about 10% to about 55% by weight, and more specifically between about 15% and about 40% by weight.

The precursor mixture that is provided is formed into a desired shape by means well known in the art. The forming process can include extrusion, pressing, pelletizing, molding, casting, etc. In one particularly embodiment, the forming includes an extrusion process. In another particular embodiment, the shape formed is a so called Raschig ring or a hollow cylinder with at least one hole. Typically, such a cylinder has an outer diameter from about 4 millimeters to about 10 millimeters, and a length about equal to the outer diameter (i.e., L is from about 4 millimeters to about 10 millimeters).

In some embodiments in which improved crush strength is desired, the porous ceramic body is formed into a cylinder comprising at least two spaced apart holes that extend through an entire length of the cylinder. By “entire length” is meant that the holes extend from a topmost surface of the cylinder to a bottommost surface of the cylinder. In one embodiment, the cylinder comprises three spaced apart holes that extend through the entire length of the cylinder. In another embodiment, the cylinder comprises five spaced apart holes that extend through the entire length of the cylinder. In a further embodiment, the cylinder comprises seven spaced apart holes that extend through the entire length of the cylinder. In such embodiments, the average crush strength of a cylindrically shaped porous body with the same length and outer diameter is significantly improved for the multi-hole cylindrically shaped porous bodies as compared to a single-hole cylindrically shaped porous body of the same material, same outer diameter and same length. The cylinders that can be employed in the present invention have an outer diameter that can range from about 1 millimeter to about 100 millimeters. Each hole that is present in the cylinder has a same inner diameter which can range from about 0.2 millimeters to about 30 millimeters. The length of the cylinder may vary depending upon the ultimate use of the shaped porous body. In one embodiment, the cylinder may have a length from about 1 millimeter to about 100 millimeters. The maximum number of holes that can be present in the cylinder is dependent on the outer diameter of the cylinder. In one embodiment, the cylinder has an outer diameter of from about 4 millimeters to about 10 millimeters, a length the is about equal to the outer diameter (i.e., L is from about 4 millimeters to about 10 millimeters) and the cylinder comprises three to twenty spaced holes that extend through the entire length of the cylinder. In such an embodiment, it is preferred that the cylinder contains 5 to 7 holes.

After completing the forming process, the formed shape is subjected to an optional drying step to remove solvents, if any, and a subsequent heat treatment step in which it is calcined or sintered to produce the porous body. The heat treatment process generally employs a temperature in a range of about 100° C. to about 2000° C. depending upon type of the alumina precursor and the desired phase(s) to be synthesized. The calcination step would also necessarily function to remove volatiles, such as water and the burnout material. However, in some embodiments, a preceding lower temperature heat treatment (also referred to herein as a “pre-calcining step”) is conducted before the calcination or sintering step in order to remove such volatiles. The preceding lower temperature heat treatment generally employs a temperature of about 35° C. to about 900° C. Generally, a heating and/or cooling rate within a range of 0.5-100° C./min, preferably 1-20° C./min, or more preferably 2-5° C./min, is used.

In one embodiment of the present invention, a porous alpha alumina body is provided. The porous alpha alumina body includes at least 80% alpha alumina crystallites in which at least 10%, more preferably, at least 50%, and even more preferably, at least 75%, of the alpha alumina crystallites are in the form of mesocrystals.

The porous alpha alumina body of the present invention typically has a pore volume up to 1.0 mL/g. In one embodiment, the porous alpha alumina body of the present invention has a pore volume from 0.2 mL/g to 1.0 mL/g. In another embodiment, the porous alpha alumina body of the present invention has a pore volume from 0.30 mL/g to 0.9 mL/g. In some embodiments of the present invention, the porous alumina body of the present invention has a water absorption from 20 percent to 100 percent, with a range from 30 percent to 90 percent being more typical.

The porous alpha alumina body of the present invention typically has a B.E.T. surface area up to 20 m²/g. In one embodiment, the surface area of the porous alpha alumina body of the present invention is from 0.4 m²/g to 3.5 m²/g. In another embodiment, the porous alpha alumina body of the present invention has a surface area from 0.5 m²/g to 1.2 m²/g. In yet another embodiment, the porous alpha alumina body of the present invention has a surface area above 1.2 m²/g up to, and including, 3.0 m²/g. The B.E.T. surface area described herein can be measured by any suitable method, but is more preferably obtained by the method described in Brunauer, S., et al., J. Am. Chem. Soc., 60, 309-16 (1938).

The porous alpha alumina body of the present invention can be monomodal, or multimodal, such as, for example, bimodal. The porous alpha alumina body of the present invention has a pore size distribution with at least one mode of pores in the range from 0.01 micrometers to 100 micrometers. In one embodiment of the present invention, at least 90 percent of the pore volume of the body is attributed to pores having a pore size of 10 microns or less. In yet another embodiment of the present invention, at least 80 percent of the pore volume of the porous alpha alumina body is attributed to pores having a size from 0.3 micron to 7 micron. In a further embodiment of the present invention, at least 80 percent of the pore volume of the porous alpha alumina body is attributed to pores having a size from 0.3 micron to 5 micron. In an even further embodiment of the present invention at least 80 percent of the pore volume of the porous alpha alumina body is attributed to pores having a size from 0.5 micron to 10 microns.

In the case of a multimodal pore size distribution, each pore size distribution can be characterized by a single mean or median pore size (mean or median pore diameter) value. Accordingly, a mean or median pore size value given for a pore size distribution necessarily corresponds to a range of pore sizes that results in the indicated mean pore size value. Any of the exemplary pore sizes given above can alternatively be understood to indicate a mean (i.e., average or weighted average) pore size. Each peak pore size can be considered to be within its own pore size distribution (mode), i.e., where the pore size concentration on each side of the distribution falls to approximately zero (in actuality or theoretically). The multimodal pore size distribution can be, for example, bimodal, trimodal, or of a higher modality. In one embodiment, different pore size distributions, each having a peak pore size, are non-overlapping by being separated by a concentration of pores of approximately zero (i.e., at baseline). In another embodiment, different pore size distributions, each having a peak pore size, are overlapping by not being separated by a concentration of pores of approximately zero.

In one embodiment, the porous alpha alumina body of the present invention may be bimodal having a first set of pores from 0.01 microns to 1 micron and a second set of pores from greater than 1 micron to 10 microns. In such an embodiment, the first set of pores may constitute less that 50 percent of the total pore volume of the porous alpha alumina body, while the second set of pores may constitute more than 50 percent of the total pore volume of the porous alpha alumina body. In another embodiment, the first set of pores may constitute more that 50 percent of the total pore volume of the porous alpha alumina body, while the second set of pores may constitute less than 50 percent of the total pore volume of the porous alpha alumina body. In yet another embodiment, the pore size distribution has a single mode between 0.5 and 4 microns.

The porous alpha alumina body of the present invention typically has a total porosity that is from 45 percent to 80 percent by volume. More typically, the porous alpha alumina body of the present invention typically has a total porosity that is from 55 percent to 78 percent.

The porous alpha alumina body of the present invention typically has an average flat plate crush strength from 30 N to 200 N. More typically, the porous alpha alumina body of the present invention typically has an average flat plate crush strength from 40 N to 150 N. The flat plate crush strength of the porous alumina bodies can be measured using a standard test method for single pellet crush strength of formed catalysts and catalyst carriers, ASTM Standard ASTM D4179.

In some embodiments, the porous alpha alumina body of the present invention can have an attrition value that is less than 40%, preferably less than 25%. In some embodiments of the present invention, the porous alpha alumina body can have attrition less that 10%. Attrition measurements of the porous alumina bodies can be performed using a standard test method for attrition and abrasion of catalysts and catalyst carriers, ASTM Standard ASTM D₄₀₅₈.

In some embodiments of the present invention, the porous alpha alumina body of the present invention has an initial low alkali metal content. By “low alkali metal content” it is meant that the porous alpha alumina body contains from 2000 ppm or less, typically from 30 ppm to 300 ppm, of alkali metal therein. Porous alpha alumina bodies containing low alkali metal content can be obtained by adding substantially no alkali metal during the porous body manufacturing process. By “substantially no alkali metal” it is meant that only trace amounts of alkali metal are used during the porous alpha alumina body manufacture process as impurities from other constituents of the porous alumina body. In another embodiment, porous alpha alumina body having a low alkali metal content can be obtained by performing various washing steps to the porous alumina body precursor materials used in forming the porous alpha alumina body. The washing steps can include washing in a base, water, or an acid.

In other embodiments of the present invention, the porous alpha alumina body has an alkali metal content that is above the value mentioned above for the porous alpha alumina body having substantially no alkali metal content. In such an embodiment, the porous alpha alumina body typically contains a measurable level of sodium on the surface thereof. The concentration of sodium at the surface of the porous alpha alumina body will vary depending on the level of sodium within the different components of the porous alpha alumina body as well as the details of its calcination. In one embodiment of the present invention, the porous alpha alumina body has a surface sodium content of from 2 ppm to 150 ppm, relative to the total mass of the porous alpha alumina body. In another embodiment of the present invention, the porous alpha alumina body has a surface sodium content of from 5 ppm to 70 ppm, relative to the total mass of the porous alpha alumina body. The sodium content mentioned above represents that which is found at the surface of the porous alpha alumina body and that which can be leached, i.e., removed, by, for example, nitric acid (hereafter referred to as acid-leachable sodium).

The quantity of acid leachable sodium present in the porous alpha alumina body of the present invention can be extracted from the catalyst or carrier with 10% nitric acid in deionized water at 100° C. The extraction method involves extracting a 10-gram sample of the catalyst or carrier by boiling it with a 100 ml portion of 10% w nitric acid for 30 minutes (1 atm., i.e., 101.3 kPa) and determining in the combined extracts the relevant metals by using a known method, for example atomic absorption spectroscopy (See, for example, U.S. Pat. No. 5,801,259 and U.S. Patent Application Publication No. 2014/0100379 A1).

In one embodiment of the present invention, the porous alpha alumina body may have a silica content, as measured as SiO₂, of less than 0.5 weight percent, and a sodium content, as measured as Na₂O, of less than 0.1 weight percent. In some embodiments, the porous alpha alumina body may have a silica content, as measured as SiO₂, of less than 0.3 weight percent. In some embodiments, the porous alpha alumina body of the present invention may have an acid leachable sodium content of 300 ppm or less.

The porous alpha alumina body of the present application can be of any suitable shape or morphology. For example, the porous alpha alumina body of the present application can be in the form of particles, chunks, pellets, rings, spheres, three-holes, wagon wheels, cross-partitioned hollow cylinders, and the like, of a size preferably suitable for employment in fixed bed reactors.

In one embodiment, the porous alpha alumina body contains essentially only alumina in the absence of other metals or chemical compounds, except that trace quantities of other metals or compounds may be present. A trace amount is an amount low enough that the trace species does not observably affect functioning or ability of the catalyst.

In some embodiments, the porous alpha alumina body (or any of the other porous ceramic bodies) of the present invention can be used as a catalyst carrier (i.e., catalyst support), in which case it typically contains one or more catalytically active species, typically metals, disposed on or in the porous body. The one or more catalytically active materials can catalyze a specific reaction and are well known in the art. In some embodiments, the catalytically active material includes one or more transition metals from Groups 3-14 of the Periodic Table of Elements and/or lanthanides. In such applications, one or more promoting species (i.e., species that aide in a specific reaction) can be also disposed on or in the porous body of the present invention. The one or more promoting species may be, for example, alkali metals, alkaline earth metals, transition metals, and/or an element from Groups 15-17 of the Periodic Table of Elements.

In one example, a porous alpha alumina body of the present invention is used as a carrier for silver-based epoxidation catalysis, the carrier includes silver on and/or in the porous alumina carrier. Thus, in the method described above, generally after the sintering step, the silver is incorporated on or into the porous alumina body by means well known in the art, e.g., by impregnation of a silver salt followed by thermal treatment, as well known in the art, as described in, for example, U.S. Pat. Nos. 4,761,394, 4,766,105, 4,908,343, 5,057,481, 5,187,140, 5,102,848, 5,011,807, 5,099,041 and 5,407,888, all of which are incorporated herein by reference. The concentration of silver salt in the solution is typically in the range from about 0.1% by weight to the maximum permitted by the solubility of the particular silver salt in the solubilizing agent employed. More typically, the concentration of silver salt is from about 0.5% by weight of silver to 45% by weight of silver, and even more typically, from about 5% by weight of silver to 35% by weight of silver by weight of the carrier. The foregoing amounts are typically also the amounts by weight found in the catalyst after thermal treatment. To be suitable as an ethylene epoxidation catalyst, the amount of silver should be a catalytically effective amount for ethylene epoxidation, which may be any of the amounts provided above.

In some embodiments, the catalytic amount of silver is up 50% by weight. In other embodiments, the catalytic amount of silver is from 10 to 50% by weight.

In addition to silver, the silver-based epoxidation catalyst of the present invention may also include any one or more promoting species in a promoting amount. The one or more promoting species can be incorporated into the porous body described above either prior to, coincidentally with, or subsequent to the deposition of the silver. As used herein, a “promoting amount” of a certain component of a catalyst refers to an amount of that component that works effectively to provide an improvement in one or more of the catalytic properties of the catalyst when compared to a catalyst not containing the component.

For example, the silver-based epoxidation catalyst may include a promoting amount of a Group I alkali metal or a mixture of two or more Group 1 alkali metals. Suitable Group 1 alkali metal promoters include, for example, lithium, sodium, potassium, rubidium, cesium or combinations thereof. Cesium is often preferred, with combinations of cesium with other alkali metals also being preferred. The amount of alkali metal will typically range from about 10 ppm to about 3000 ppm, more typically from about 15 ppm to about 2000 ppm, more typically from about 20 ppm to about 1500 ppm, and even more typically from about 50 ppm to about 1000 ppm by weight of the total catalyst, expressed in terms of the alkali metal.

The silver-based epoxidation catalyst may also include a promoting amount of a Group 2 alkaline earth metal or a mixture of two or more Group 2 alkaline earth metals. Suitable alkaline earth metal promoters include, for example, beryllium, magnesium, calcium, strontium, and barium or combinations thereof. The amounts of alkaline earth metal promoters are used in similar amounts as the alkali metal promoters described above.

The silver-based epoxidation catalyst may also include a promoting amount of a main group element or a mixture of two or more main group elements. Suitable main group elements include any of the elements in Groups 13 (boron group) to 17 (halogen group) of the Periodic Table of the Elements. In one example, a promoting amount of one or more sulfur compounds, one or more phosphorus compounds, one or more boron compounds or combinations thereof can be used.

The silver-based epoxidation catalyst may also include a promoting amount of a transition metal or a mixture of two or more transition metals. Suitable transition metals can include, for example, the elements from Groups 3 (scandium group), 4 (titanium group), 5 (vanadium group), 6 (chromium group), 7 (manganese group), 8-10 (iron, cobalt, nickel groups), and 11 (copper group) of the Periodic Table of the Elements, as well as combinations thereof. More typically, the transition metal is an early transition metal selected from Groups 3, 4, 5, 6, or 7 of the Periodic Table of Elements, such as, for example, hafnium, yttrium, molybdenum, tungsten, rhenium, chromium, titanium, zirconium, vanadium, tantalum, niobium, or a combination thereof.

In one embodiment of the present invention, the silver-based epoxidation catalyst includes silver, cesium, and rhenium. In another embodiment of the present invention, the silver-based epoxidation catalyst includes silver, cesium, rhenium and one or more species selected from Li, K, W, Zn, Mo, Mn, and S.

The silver-based epoxidation catalyst may also include a promoting amount of a rare earth metal or a mixture of two or more rare earth metals. The rare earth metals include any of the elements having an atomic number of 57-71, yttrium (Y) and scandium (Sc). Some examples of these elements include lanthanum (La), cerium (Ce), and samarium (Sm).

The transition metal or rare earth metal promoters are typically present in an amount of from about 0.1 micromoles per gram to about 10 micromoles per gram, more typically from about 0.2 micromoles per gram to about 5 micromoles per gram, and even more typically from about 0.5 micromoles per gram to about 4 micromoles per gram of total catalyst, expressed in terms of the metal. All of the aforementioned promoters, aside from the alkali metals, can be in any suitable form, including, for example, as zerovalent metals or higher valent metal ions.

The silver-based epoxidation catalyst may also include an amount of rhenium (Re), which is known as a particularly efficacious promoter for ethylene epoxidation high selectivity catalysts. The rhenium component in the catalyst can be in any suitable form, but is more typically one or more rhenium-containing compounds (e.g., a rhenium oxide) or complexes. The rhenium can be present in an amount of, for example, about 0.001 wt. % to about 1 wt. %. More typically, the rhenium is present in amounts of, for example, about 0.005 wt. % to about 0.5 wt. %, and even more typically, from about 0.01 wt. % to about 0.10 wt. % based on the weight of the total catalyst including the support, expressed as rhenium metal. All of these promoters, aside from the alkali metals, can be in any suitable form, including, for example, as zerovalent metals or higher valent metal ions.

After impregnation with silver and any promoters, the impregnated carrier is removed from the solution and calcined for a time sufficient to reduce the silver component to metallic silver and to remove volatile decomposition products from the silver-containing support. The calcination is typically accomplished by heating the impregnated carrier, preferably at a gradual rate, to a temperature in a range of about 200° C. to about 600° C., more typically from about 200° C. to about 500° C., more typically from about 250° C. to about 500° C., and more typically from about 200° C. or 300° C. to about 450° C., at a reaction pressure in a range from about 0.5 to about 35 bar. In general, the higher the temperature, the shorter the required calcination period. A wide range of heating periods have been described in the art for the thermal treatment of impregnated supports. See, for example, U.S. Pat. No. 3,563,914, which indicates heating for less than 300 seconds, and U.S. Pat. No. 3,702,259, which discloses heating from 2 to 8 hours at a temperature of from 100° C. to 375° C. to reduce the silver salt in the catalyst. A continuous or step-wise heating program may be used for this purpose. During calcination, the impregnated support is typically exposed to a gas atmosphere comprising an inert gas, such as nitrogen. The inert gas may also include a reducing agent.

In another embodiment, the porous ceramic body of the present invention can be used as a filter in which liquid or gas molecules can diffuse through the pores of the porous ceramic body described above. In such an application, the porous ceramic body of the present invention be placed along any portion of a liquid or gas stream flow. In yet another embodiment of the present invention, the porous ceramic body of the present invention can be used as a membrane. In another embodiment, the porous ceramic body of the present invention can be used as an insulator, a refractory, a filler, an abrasive, or as a substrate.

In another aspect, the present invention is directed to a method for the vapor phase production of ethylene oxide by conversion of ethylene to ethylene oxide in the presence of oxygen by use of the silver-based epoxidation catalyst described above. Generally, the ethylene oxide production process is conducted by continuously contacting an oxygen-containing gas with ethylene in the presence of the catalyst at a temperature in the range from about 180° C. to about 330° C., more typically from about 200° C. to about 325° C., and more typically from about 225° C. to about 270° C., at a pressure which may vary from about atmospheric pressure to about 30 atmospheres depending on the mass velocity and productivity desired. Pressures in the range of from about atmospheric to about 500 psi are generally employed. Higher pressures may, however, be employed within the scope of the invention. Residence times in large-scale reactors are generally on the order of about 0.1 to about 5 seconds. A typical process for the oxidation of ethylene to ethylene oxide comprises the vapor phase oxidation of ethylene with molecular oxygen in the presence of the inventive catalyst in a fixed bed, tubular reactor. Conventional commercial fixed bed ethylene oxide reactors are typically in the form of a plurality of parallel elongated tubes (in a suitable shell). In one embodiment, the tubes are approximately 0.7 to 2.7 inches O.D. and 0.5 to 2.5 inches I.D. and 15-45 feet long filled with catalyst.

In some embodiments, the silver-based epoxidation catalyst described above exhibits a high level of selectivity in the oxidation of ethylene with molecular oxygen to ethylene oxide. For example, a selectivity value of at least about 83 mol % up to about 93 mol % may be achieved. In some embodiments, the selectivity is from about 87 mol % to about 93 mole %. The conditions for carrying out such an oxidation reaction in the presence of the silver-based epoxidation catalyst described above broadly comprise those described in the prior art. This applies, for example, to suitable temperatures, pressures, residence times, diluent materials (e.g., nitrogen, carbon dioxide, steam, argon, and methane), the presence or absence of moderating agents to control the catalytic action (e.g., 1, 2-dichloroethane, vinyl chloride or ethyl chloride), the desirability of employing recycle operations or applying successive conversion in different reactors to increase the yields of ethylene oxide, and any other special conditions which may be selected in processes for preparing ethylene oxide.

In the production of ethylene oxide, reactant feed mixtures typically contain from about 0.5 to about 45% ethylene and from about 3 to about 15% oxygen, with the balance comprising comparatively inert materials including such substances as nitrogen, carbon dioxide, methane, ethane, argon and the like. Only a portion of the ethylene is typically reacted per pass over the catalyst. After separation of the desired ethylene oxide product and removal of an appropriate purge stream and carbon dioxide to prevent uncontrolled build up of inert products and/or by-products, unreacted materials are typically returned to the oxidation reactor.

It is noted that silver-based EO catalysts that are supported on carrier composed of the porous alpha alumina body containing a mesocrystal microstructure, as described in the present invention, exhibit enhanced activity and catalyst lifetime compared to an equivalent EO catalyst supported on a carrier composed of a porous alpha alumina body that lacks the mesocrystal microstructure. The aspect of the present invention will be exemplified in the examples of the present invention.

Examples have been set forth below for the purpose of further illustrating the present invention. The scope of the present invention is not to be in any way limited by the examples set forth herein.

### Example 1: Porous Alpha Alumina Body Preparation

In this example, seven different porous alpha alumina bodies in accordance with the present application, namely, PB1, PB2, PB3, PB4, PB5, PB6 and PB7, were prepared, together with two comparative porous alpha alumina bodies, namely, CPB1 and CPB2. The inventive porous alpha alumina bodies namely, PB1, PB2, PB3, PB4, PB5, PB6 and PB7, were prepared from a precursor mixture composition as shown in Table 1 below. The inventive porous alumina bodies were prepared utilizing one of the precursor mixture preparation processes mentioned above and include materials that are also mentioned above. CP1 was also prepared utilizing the same precursor mixture preparation process as the inventive porous bodies.

Some of the precursor mixtures of the present invention did form flowable powders and/or granules. Other examples of the precursor mixtures of the present invention did not form flowable media. However, all of the precursor mixtures of the present invention were mixed and homogenized fairly well without major inhomogenities.

Subsequently, each of the precursor mixtures of the present invention was extruded using 2″ Bonnot extruder with a single die to produce extrudate in the shape of hollow cylinders. The extrudates were cut into equal-length pieces and then dried under a heat lamp for 1 hr.

Subsequently, the cut and dried extrudates were moved to a furnace and subjected to the following heat treatments: (i) pyrolysis of the organic components (burnout) was performed in flowing air at 100° C.-900° C. for 1-24 hrs, followed by (ii) sintering at 1200° C.-1600° C. for 3-12 hrs with the heating rates of 2.0-5.0° C./min.

CPB1 was prepared utilizing the general methodology described above. CPB2 was made using a different methodology, not of the present invention.

TABLE 1
COMPOSITION OF DIFFERENT POROUS ALPHA ALUMINA BODIES

| Precursor | | | | | Solvents and | | | |
|---|---|---|---|---|---|---|---|---|
| Formulation for | Alumina | Alumina | Alumina | | Lubricants | Burn- | Burn- | Burn- |
| porous alpha | Powder 1 | Powder 2 | Powder 3 | Binder | Total | out 1 | out 2 | out 3 |
| alumina body | (kg) | (kg) | (kg) | (kg) | (kg) | (kg) | (kg) | (kg) |
| PB1 | 2.5 | — | — | 0.08 | 1.5 | 1.0 | 0.5 | — |
| PB2 | 3.3 | — | 0.035 | 0.08 | 2.5 | 0.4 | 0.8 | — |
| PB3 | — | 3.46 | — | 0.10 | 1.4 | 0.7 | 1.4 | — |
| PB4 | — | 3.46 | 0.035 | 0.10 | 1.4 | 0.7 | 1.4 | — |
| PB5 | 2.5-3.3 | — | 0-0.035 | 0.06-0.08 | 1.0-2.8 | 0.2-2.0 | 0.0-0.8 | 0.0-1.2 |
| PB6 | 1.6-3.3 | 0.2-1.3 | 0.035-0.250 | 0.06-0.08 | 1.8-2.5 | 0.2-0.4 | 0.6-0.8 | — |
| PB7 | — | 1.5-2.5 | 0-0.025 | 0.05-0.10 | 0.8-3.5 | 0.0-2.5 | 0.0-2.5 | 0.0-2.5 |
| CPB1 | — | 0.6-1.2 | 1.5-2.1 | — | 1.5-2.4 | 0.4-1.1 | 0.9-1.8 | — |

| | |
|---|---|
| CPB2 | This comparative porous body was made using a different methodology, not of the present invention |

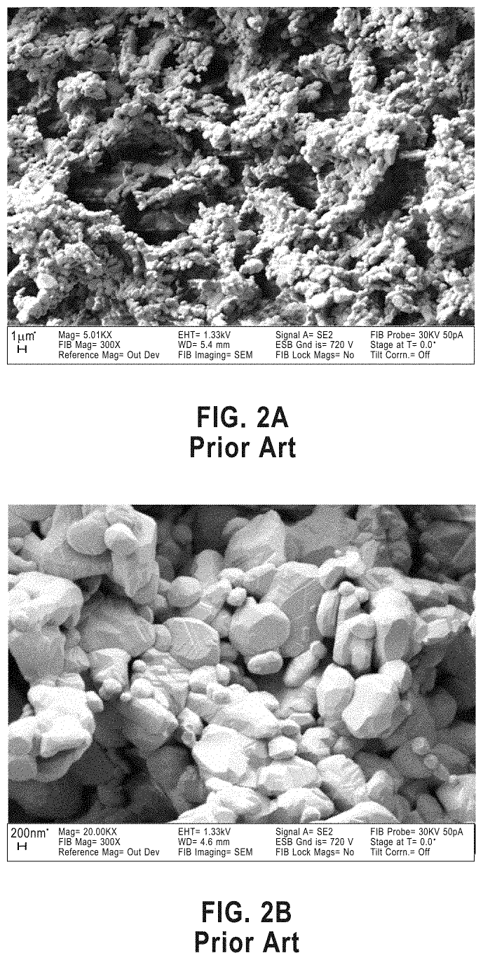

Reference is now made to FIG. 2A (at a magnification of 5,000×, scale-bar 1 micron) and FIG. 2B (at a magnification of 20,000×, scale-bar 200 nm) which are scanning electron microscopy (SEM) images of CPB1. CPB1 was made using a precursor mixture of primarily equiaxed alpha alumina crystallites, also with clearly visible faceting and grain boundaries.

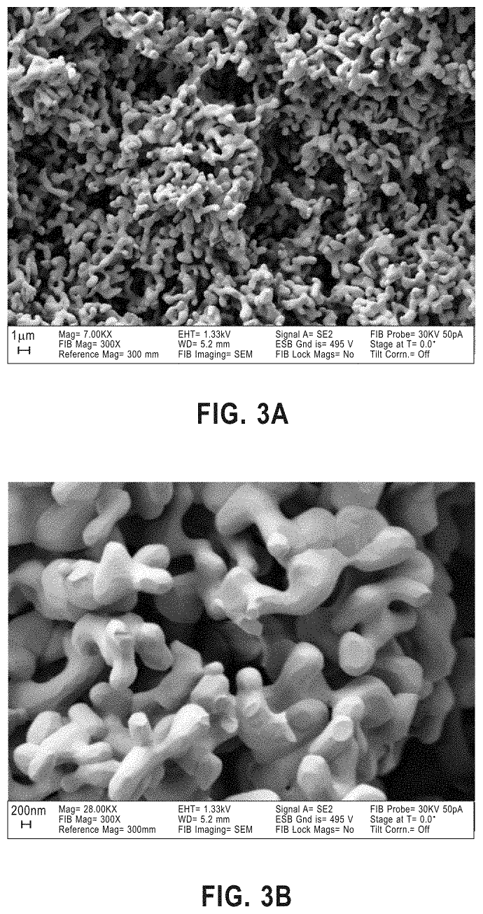

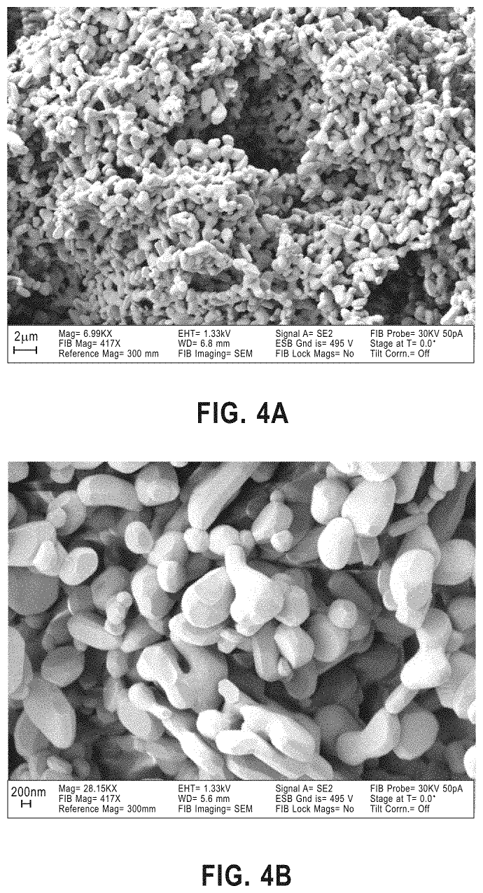

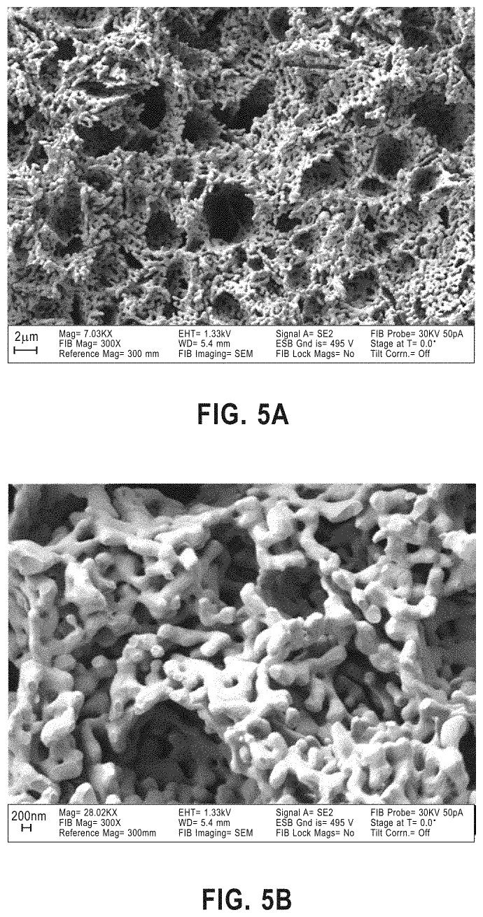

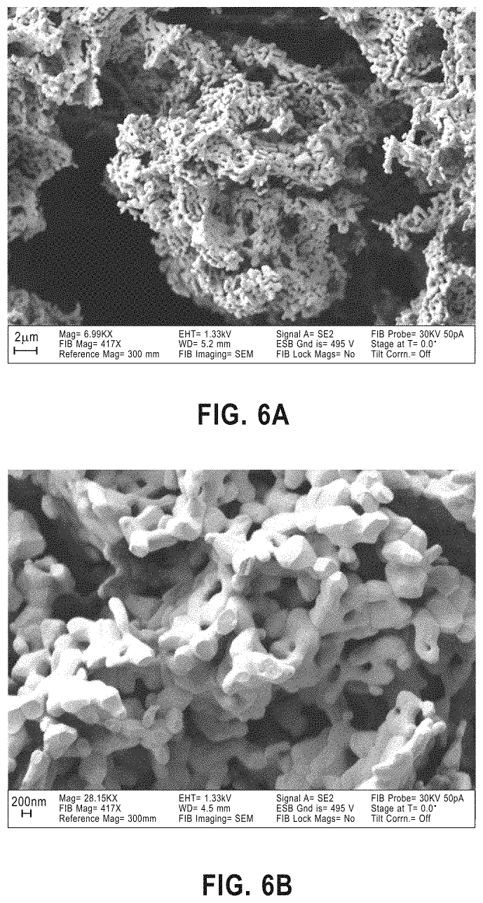

Reference is now made to the SEM images shown in FIGS. 3A, 3B, 4A, 4B, 5A, 5B, 6A and 6B. Notably, FIGS. 3A-3B are SEM images of PB1, FIGS. 4A-4B are SEM images of PB2, FIGS. 5A-5B are SEM images of PB3, and FIGS. 6A-6B are SEM images of PB4. Each of the ‘A’ labeled SEM pictures are at magnification of 7,000× and with scale bar of 2 microns (except FIG. 3A that has a scale bar of 1 micron), while each of the “B’ labeled SEM pictures are at magnification of 28,000× with scale bar of 200 nm). The porous alpha alumina bodies of the present invention, including PB1, PB2, PB3 and PB4 exemplified in the SEM images, include non-faceted alpha alumina crystallites forming worm-like and well-branched structures. Grain boundaries are in some cases visible but clearly less frequent. Size of the crystallites, their connectivity and shapes vary with the synthesis method and are slightly different for each of the inventive porous alpha alumina bodies, including PB1, PB2, PB3 and PB 4 exemplified in the SEM pictures. It should be noted that there are occasional flat surfaces visible on the SEM images but they are not necessarily from crystallographic faceting of alpha alumina crystallites. These occasional flat regions are in most cases fracture surfaces of the porous alpha alumina body, which had to be broken prior to the SEM measurements.

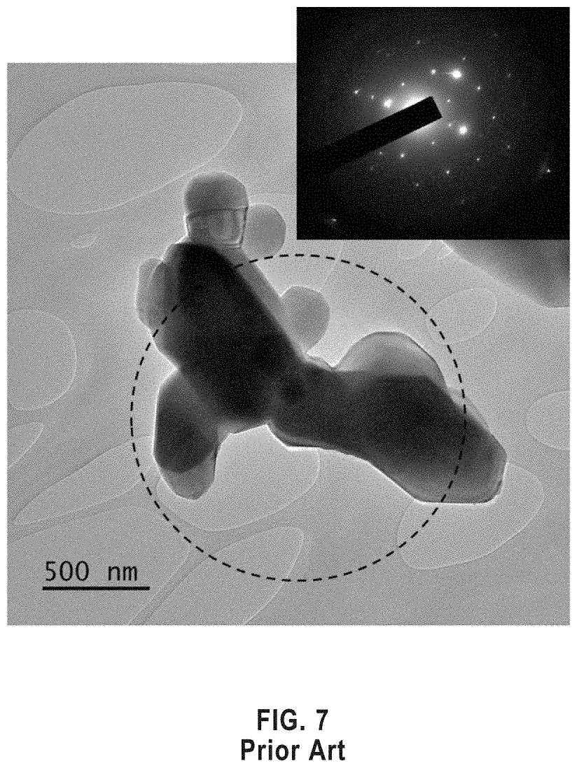

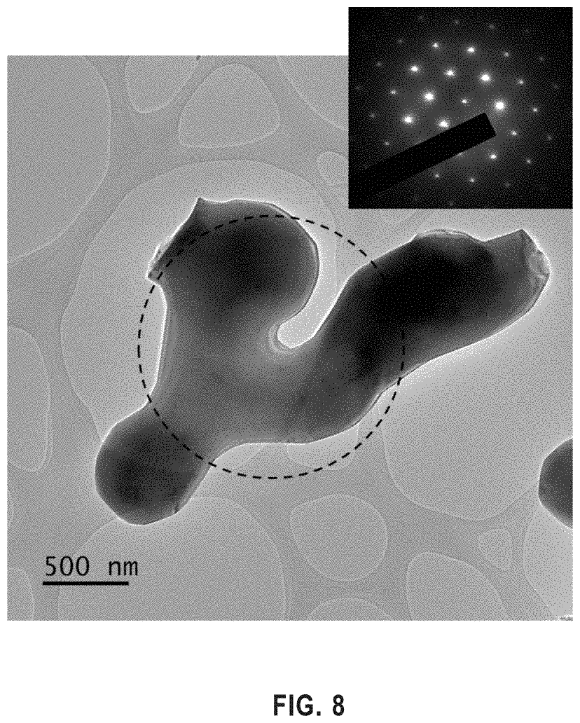

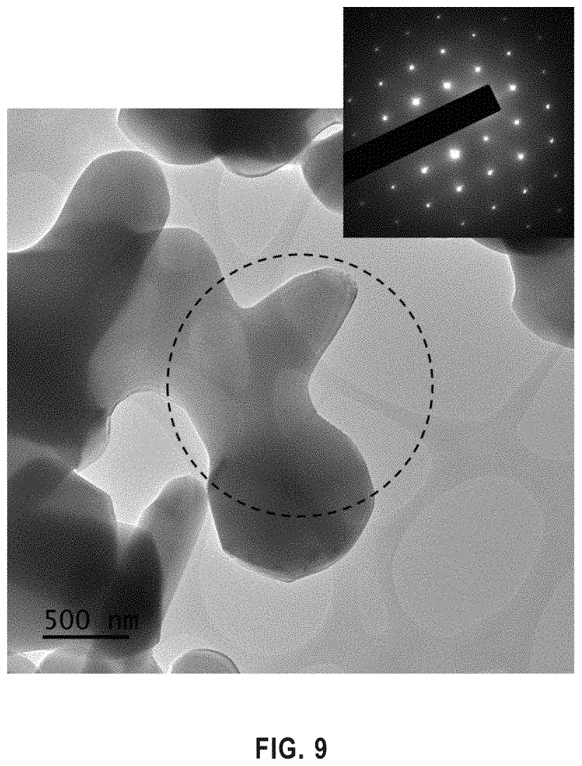

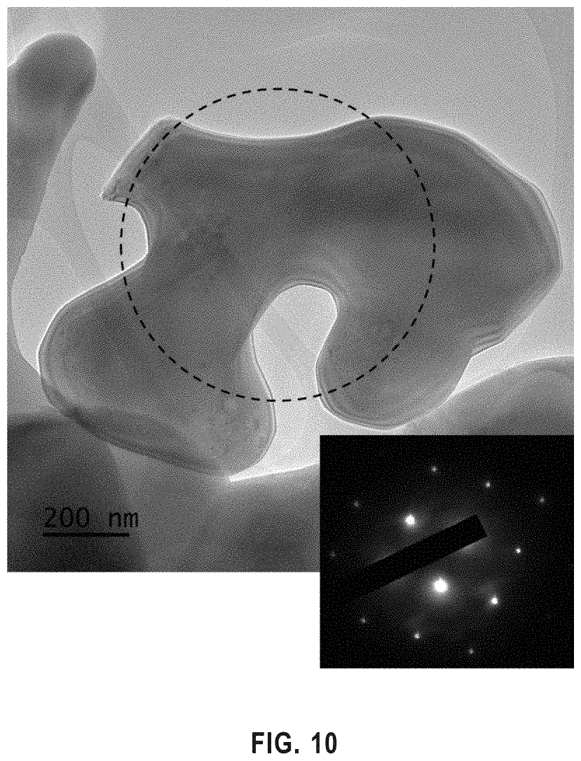

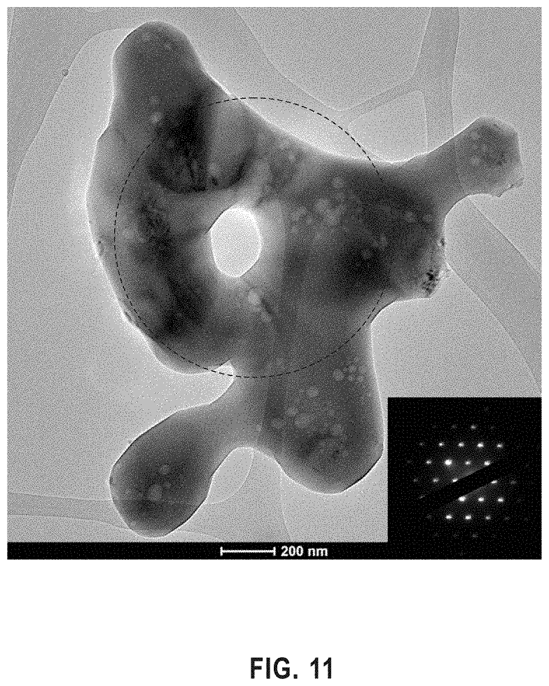

Reference is now made to the transmission electron microscopy (TEM) images shown in FIGS. 7, 8, 9, 10, and 11, with insets containing electron diffraction patterns of each of the exemplified porous alpha alumina bodies. Notably, FIG. 7 is the TEM image of CPB1, with an insert including the electron diffraction pattern of CPB1, FIG. 8 is the TEM image of PB1, with an insert including the electron diffraction pattern of PB1, FIG. 9 is the TEM image of PB2, with an insert including the electron diffraction pattern of PB2, FIG. 10 is the TEM image of PB4, with an insert including the electron diffraction pattern of PB4 and FIG. 11 is the TEM image of PB7, with an insert including the electron diffraction pattern of PB7. In brief, the detailed TEM analysis of the non-faceted alpha alumina crystallites confirmed lack of clear faceting and grain boundaries within small regions of crystallites. Fracture surfaces were visible. Electron diffraction confirmed the same crystallographic orientation of all presented crystallite regions. Such types of polycrystalline structures are called mesocrystals.

The TEM image of CPB1 shown in FIG. 7 shows that CPB1 had a polycrystalline appearance. The polycrystalline appearance is confirmed by the accompanying electron diffraction pattern (see, inset of FIG. 7), which is from at least two separate single crystals. Grain boundaries and crystal faceting can be clearly visible. The area used for electron diffraction is marked by the dashed circle.

The TEM image of PB1 shown in FIG. 8 shows that PB1 had an apparent polycrystalline appearance. Despite the apparent polycrystalline appearance of PB1, the electron diffraction pattern is of a single crystal (i.e., mesocrystal). No clear grain boundaries are visible, confirming mesocrystalline nature of PB1 without characteristic crystallographic facets. The area used for electron diffraction is marked by the dashed circle.

The TEM image of PB2 shown in FIG. 9 shows that PB2 had an apparent polycrystalline appearance. Despite the apparent polycrystalline appearance of PB2, the electron diffraction pattern is of a single crystal (i.e., mesocrystal). No clear grain boundaries are visible, confirming mesocrystalline nature of PB2 without characteristic crystallographic facets. The area used for electron diffraction is marked by the dashed circle.

The TEM image of PB4 shown in FIG. 10 shows that PB4 had an apparent polycrystalline appearance. Despite the apparent polycrystalline appearance of PB2, the electron diffraction pattern is of a single crystal (i.e., mesocrystal). No clear grain boundaries are visible, confirming mesocrystalline nature of PB4 without characteristic crystallographic facets. The area used for electron diffraction is marked by the dashed circle.

The TEM image of PB7 shown in FIG. 11 shows that PB7 had an apparent polycrystalline appearance. Despite the apparent polycrystalline appearance of PB7, the electron diffraction pattern is of a single crystal (i.e., mesocrystal). No clear grain boundaries are visible, confirming mesocrystalline nature of PB7 without characteristic crystallographic facets. The area used for electron diffraction is marked by the dashed circle.

### Example 2: Physical and Chemical Properties of Porous Alpha Alumina Bodies

The physical properties of all the porous alpha alumina bodies prepared in Example 1 were characterized using standard methodology as is described in the present application. Table 2 below summarizes the physically properties of each of the porous alpha alumina bodies prepared in Example 1.

TABLE 2
PHYSICAL PROPERTIES OF POROUS ALUMINA BODIES

| | Pore | BET | Average Flat | Attrition (%) |
|---|---|---|---|---|
| Porous ALPHA | Volume | Surface Area | Plate Crush | (selected |
| Alumina Body | (mL/g) | (m²/g) | Strength (N) | samples only) |
| PB1 | 0.45-0.68 | 0.9-1.5 | 50-80 | 18 |
| PB2 | 0.55-0.62 | 1.3-1.5 | 45-75 | 8 |
| PB3 | 0.64-0.73 | 2.0-3.1 | 90-110 | 2 |
| PB4 | 0.52-0.61 | 1.5-2.1 | 79-94 | 5 |
| PB5 | 0.31-0.86 | 0.8-2.3 | 34-117 | 7-21 |
| PB6 | 0.46-0.77 | 1.0-1.7 | 38-111 | 9-20 |
| PB7 | 0.32-0.72 | 0.9-3.7 | 29-224 | 5-25 |
| CPB1 | 0.66-0.84 | 0.8-1.2 | 50-80 | 16-21 |
| CPB2 | 0.32-0.60 | 0.4-1.0 | 80-150 | 20-25 |

In general, the porous alpha alumina bodies, PB1-PB7, of the present application have higher BET and much lower attrition than the comparative porous alpha alumina bodies, CPB1. The pore volumes and crush strength encompass a broad range of values. Median pore diameters of the porous alumina bodies, PB1-PB4, of the present application are between 0.8 and 3 μm without any pores smaller than 0.3 μm. The distributions are single or bi-modal.

Typical porous alpha alumina bodies, PB1-PB7, of the present application have the following level of bulk impurities (XRF, GDMS): [SiO₂]=0.005-0.1 wt %, [Na₂O]=0.002-0.05 wt %, [CaO]=0.005-0.3 wt %, [MgO]=0.005-0.1 wt %. Acid leachables components in 10% HNO₃ are: [Na]=0-300 ppm (preferred 0-100 ppm), [Si]=20-300 ppm (preferred 0-100 ppm), [Ca]=3-140 ppm, and [Mg]<20 ppm (see Table 3).

TABLE 3
RANGES OF ACID LEACHABLES
FOR POROUS ALUMINA BODIES

| Porous Alpha | Acid Leachable (ppm) in 10% HNO₃ (aq) | | | | |
|---|---|---|---|---|---|
| Alumina Bodies | [Na] | [K] | [Mg] | [Ca] | [Si] |
| PB1 | 35 | 1 | 0 | 57 | 42 |
| PB2 | 151 | 16 | 1 | 100 | 204 |
| PB3 | 1 | 1 | 1 | 20 | 30 |
| PB4 | 3 | 1 | 1 | 18 | 35 |
| PB5 | 0-250 | 0-20 | 0-3 | 8-110 | 20-300 |
| PB6 | 30-300 | 0-30 | 0-8 | 3-107 | 20-300 |
| PB7 | 0-115 | 0-43 | 3-61 | 40-324 | 20-210 |
| CPB1 | 3-70 | 0-10 | 1-20 | 30-200 | 50-300 |

| | |
|---|---|
| CPB2 | Not available |

### Example 3: Catalyst Preparation and Catalyst Performances

In this example, silver-based ethylene oxide catalysts were prepared using PB1, PB2, CPB1 and CPB2 as described above. Each of the porous alpha alumina bodies used in this example were washed prior to introducing silver and the other promoters to the carrier. Each silver-based ethylene oxide catalyst was prepared using a same catalyst impregnation technique that included a silver stock solution, and appropriate promoters, as described herein below. The catalyst composition that was provided to the porous alumina body was optimized on each particular porous alpha alumina body to yield maximum performance, i.e. combination of highest selectivity and highest activity. Only optimized catalysts were compared in the epoxidation of ethylene to ethylene oxide.

Silver Stock Solution for silver-based ethylene oxide catalysts: 277.5 g of deionized water was placed in cooling bath to maintain temperature during the whole preparation under 50° C. At continuous stirring, 221.9 g of ethylenediamine was added in small portions to avoid overheating. 174.1 g of oxalic acid dihydrate was then added to the water-ethylenediamine solution in small portions. After all oxalic acid was dissolved, 326.5 g of high purity silver oxide was added to solution in small portions. After all silver oxide was dissolved and the solution was cooled to about 35° C. it was removed from the cooling bath. After filtration, the solution contained roughly 30 wt % silver, and had a specific gravity of 1.55 g/mL.

Catalyst Preparation: A 300 g portion of one of the porous alumina bodies mentioned above, i.e., PB1, PB2, CPB1, or CPB2, was placed in a flask and evacuated to about 0.1 torr prior to impregnation. To the above silver solution were added aqueous solutions of promoters including cesium (Cs) as cesium hydroxide, rhenium (Re) as perrhenic acid, and at least one other alkali metal as hydroxide in sufficient concentrations to prepare a catalyst composition in which the Cs content in the final catalyst was from 0 ppm to 1800 ppm, the rhenium content in the final catalyst was from 0 ppm to 900 ppm, and the silver (Ag) content was between 10 and 30 percent by weight. After thorough mixing, the promoted silver solution was aspirated into the evacuated flask to cover the carrier while maintaining the pressure at about 0.1 torr. The vacuum was released after about 5 minutes to restore ambient pressure, hastening complete penetration of the solution into the pores. Subsequently, the excess impregnation solution was drained from the impregnated carrier.

Calcination of the wet catalyst was performed on a moving belt calciner. In this unit, the wet catalyst was transported on a stainless steel belt through a multi-zone furnace. All zones of the furnace were continuously purged with pre-heated, nitrogen and the temperature was increased gradually as the catalyst passed from one zone to the next. The heat supplied to the catalyst was radiated from the furnace walls and from the preheated nitrogen. In this example, the wet catalyst entered the furnace at ambient temperature. The temperature was then increased gradually to a maximum of about 300-600° C. as the catalyst passed through the heated zones. In the last (cooling) zone, the temperature of the now calcined catalyst was immediately lowered to less than 100° C. before it emerged into ambient atmosphere. The total residence time in the furnace was between 30 and 60 minutes.

The performances of the silver-based ethylene oxide catalysts containing PB1 and PB2 reveals clear activity advantage over both silver-based ethylene oxide catalysts containing CPB1 and CPB2 at similar selectivity levels. This difference is visible both at the beginnings of the runs (500 hrs on stream equivalent to 0.2 MT EO/m³ cat) and towards the end of testing at 2,000 and 3,000 hrs on stream (equivalent to 0.7 and 1.1 MT EO/m³ cat, respectively). Details of the testing conditions, selectivities and temperatures are summarized in Table 4.

TABLE 4
PERFORMANCE OF SILVER-BASED CATALYSTS
ON SELECTED POROUS ALUMINA BODIES

| | | | | Catalyst Performance | |
|---|---|---|---|---|---|
| | | Catalyst Performance | | after ~3,000 hours on stream | |
| Porous Alpha | | after 500 hours on stream | | (2,000 hours on stream) | |
| Alumina Body | Test Conditions | Selectivity | Activity | Selectivity | Activity |
| PB1 | Ground catalysts | 88% | 239° C. | 88% | 247° C. |
| | charged to laboratory | | | (88%) | (244° C.) |
| PB2 | micro-reactors | 88% | 241° C. | — | — |
| | | | | (88%) | (250° C.) |
| CPB1 | | 90% | 245° C. | 89% | 255° C. |
| | | | | (90%) | (253° C.) |
| CPB2 | | 89% | 250° C. | 88% | 265° C. |
| | | | | (89%) | (261° C.) |

While there have been shown and described what are presently believed to be the preferred embodiments of the present invention, those skilled in the art will realize that other and further embodiments can be made without departing from the spirit and scope of the invention described in this invention, and this invention includes all such modifications that are within the intended scope of the claims set forth herein.

What is claimed is:

1. A porous ceramic body comprising mesocrystals of alumina.

2. The porous ceramic body of claim 1, wherein the alumina comprises alpha alumina (α-Al₂O₃), gamma alumina (γ-Al₂O₃), beta alumina (β-Al₂O₃), theta alumina (Θ-Al₂O₃), delta alumina (δ-Al₂O₃), chi alumina (χ-Al₂O₃), rho alumina (ρ-Al₂O₃), eta alumina (η-Al₂O₃) transition aluminas, akdalaite (5Al₂O₃.H₂O), tohdite (5Al₂O₃.H₂O), boehmite (γ-AlOOH), pseudo-boehmite (AlOOH), diaspore (α-Al(OH)₃), gibbsite (α-Al(OH)₃), hydrargillite (α-Al(OH)₃), bayerite (β-Al(OH)₃), doyleite (Al(OH)₃), nordstrandite (Al(OH)₃), amorphous aluminas, and mixtures thereof.

3. The porous ceramic body of claim 1, wherein the alumina comprises at least 80% alpha alumina, and the porous ceramic body has a pore volume up to 1.0 mL/g, and a surface area up to 20 m²/g.

4. The porous ceramic body of claim 3, wherein at least 90 percent of the pore volume is attributed to pores having a pore size of 10 microns or less.

5. The porous ceramic body of claim 3, wherein at least 80 percent of the pore volume is attributed to pores having a size from 0.3 micron to 7 microns.

6. The porous ceramic body of claim 3, further comprising a silica content, as measured as SiO₂, of less than 0.5 weight percent, and a sodium content, as measured as Na₂O, of less than 0.1 weight percent.

7. The porous ceramic body of claim 3, further having an acid leachable sodium content of 300 ppm or less.

8. A porous alpha alumina body comprising at least 80% alpha alumina crystallites in which at least 5% of the alpha alumina crystallites are in the form of mesocrystals.

9. The porous alpha alumina body of claim 8, wherein the porous alpha alumina body has a pore volume up to 1.0 mL/g, and a surface area up to 20 m²/g.

10. The porous alpha alumina body claim 9, wherein at least 90 percent of the pore volume is attributed to pores having a pore size of 10 microns or less.

11. The porous alpha alumina body of claim 9, wherein at least 80 percent of the pore volume is attributed to pores having a size from 0.3 micron to 7 microns.

12. The porous alpha alumina body of claim 9, further comprising a silica content, as measured as SiO₂, of less than 0.5 weight percent, and a sodium content, as measured as Na₂O, of less than 0.1 weight percent.

13. The porous alpha alumina body of claim 9, further having an acid leachable sodium content of 300 ppm or less.

14. A porous ceramic body comprising alumina crystallites without well-defined crystallographic facets.

15. A silver-based epoxidation catalyst comprising:
a carrier comprising at least 80% alpha alumina, wherein the alpha alumina contains alpha alumina mesocrystals;
a catalytic amount of silver disposed on and/or in the carrier; and
a promoting amount of one or more promoters disposed on the carrier.

16. The silver-based ethylene epoxidation catalyst of claim 15, wherein the one or more promoters comprise Group 1 alkali metal promoters, one or more transition metals, one or more Group 2 alkaline earth metals or any combination thereof.

17. The silver-based ethylene epoxidation catalyst of claim 16, wherein the one or more transition metals are selected from the group consisting of Groups 4-10 of the Periodic Table of the Elements.

18. The silver-based ethylene epoxidation catalyst of claim 17, wherein the one or more transition metals are selected from the group consisting of molybdenum, rhenium, tungsten, chromium, titanium, hafnium, zirconium, vanadium, thorium, tantalum, and niobium.

19. The silver-based ethylene epoxidation catalyst of claim 17, wherein the one or more transition metals comprise rhenium, molybdenum, tungsten, or any combination thereof.

20. The silver-based ethylene epoxidation catalyst of claim 16, wherein the Group 1 alkali metal promoters are selected from the group consisting of cesium, lithium, sodium, potassium, and rubidium.

21. The silver-based ethylene epoxidation catalyst of claim 15, wherein the Group 1 alkali metal promoters comprise lithium and cesium.

22. The silver-based ethylene epoxidation catalyst of claim 15, wherein the one or more promoters comprises a promoting combination of rhenium, cesium and lithium.

23. The silver-based ethylene epoxidation catalyst of claim 15, wherein the catalytic amount of silver is up to 50% by weight.

24. A catalyst composition comprising:
a carrier comprising mesocrystals of alumina; and
a catalytic amount of at least one catalytically active material disposed on and/or in the carrier.

25. A method of forming a porous ceramic body, the method comprising:
(i) providing a precursor mixture;
(ii) forming the precursor mixture into a desired shape; and
(iii) subjecting the shaped precursor mixture to a heat treatment process to provide a porous ceramic body that contains mesocrystals of alumina.
